(19) United States
(12) Patent Application Publication
(10) Pub. No.: US 2010/0281555 A1
(43) Pub. Date: Nov. 4, 2010

(54) METHOD FOR MARKING BIO-INFORMATION INTO GENOME OF ORGANISM AND ORGANISM MARKED WITH THE BIO-INFORMATION

(75) Inventors: Myung-Dong Kim, Gangwon-Do Chuncheon (KR); Hyun-Ju Eom, Chungcheongbuk-Do Cheongwon-Gun (KR); Eun-Hee Park, Gangwon-Do Chuncheon (KR); Nam-Soo Han, Chungcheongbuk-Do Cheongju (KR)

Correspondence Address: EDWARDS ANGELL PALMER & DODGE LLP, P.O. BOX 55874, BOSTON, MA, 02205, US

(73) Assignees: Chungbuk National University Industry-Academic Cooperation Foundation, Cheongju Chungcheongbuk-Do (KR); Knu-Industry Cooperation Foundation, Chuncheon Gangwon-Do (KR)

(21) Appl. No.: 12/746,452

(22) PCT Filed: Dec. 3, 2008
(86) PCT No.: PCT/KR2008/007147

§ 371 Date: Jun. 4, 2010

(30) Foreign Application Priority Data
Dec. 4, 2007 (KR) 10-2007-0124701

(51) Int. Cl. C12N 15/63; C12N 5/10; C12N 1/21; C12N 1/15; C12Q 1/68; A01H 1/00

(52) U.S. Cl. 800/21; 435/325; 435/252.1; 435/254.1; 435/410; 435/6; 435/473; 435/455; 435/468; 800/291

(57) ABSTRACT

Disclosed herein is a method for marking bio-information into the genome of an organism, an organism marked with inherent bio-information by the method and a method for reading the bio-information. The method is characterized in that inherent bio-information encoded to a DNA sequence or RNA sequence is inserted into genome of organisms through a gene delivery system. Inherent bio-information is inserted into genome of organisms, thus avoiding loss by cell culture or artificial manipulation and being widely used even for organisms whose host-vector system is not provided. Based on these features, the method has the following advantages. First, inherent bio-informtion can be clearly obtained from the organisms themselves, rather than an additional means such as catalogs. Second, when organisms developed through desperate efforts are stolen, they can be tracked down and identified. Third, when serious problems occur by overuse or misuse of organisms, the origin thereof can be clearly determined.

### TECHNICAL FIELD

The present invention relates to a method for marking inherent bio-information by inserting the same into an organism, an organism inserted with inherent bio-information by the method and a method for reading the bio-information.

### BACKGROUND ART

The recent development of biotechnology has brought about development of a variety of organisms with useful properties, compared to conventional organisms. Examples of organisms include cell unit-type organisms such as microorganisms, animal and plant cells and organisms composed of a great deal of cells such as higher microorganisms, animals and plants.

Since microrganims have been widely used in conventional fermented foods, they are widely utilized in a variety of applications including treatment of waste water, production of medicinal proteins and production of various useful materials, etc.

Animal cells are generally used for production of proteins and antibodies targetting the human body to which microorganisms cannot be applied. Lab animals such as mice are generally utilized in research on human disesases such as cancer.

Plant cells are utilized for production of a variety of plant-derived novel compounds exhibiting superior physiological activity, and a number of plants armed with resistance to weeds or cold damage by gene recombination have been developed.

However, it is not easy to identify these transformed organisms from their parent organisms in appearance. For this reason, it is difficult to rapidly and clearly determine the actual identity of transformed organisms. This disadvantage causes a variety of problems as follows.

First, when an organism, developed through desperate and extensive efforts, is stolen, tracking down the stolen organism, and identifying whether or not the suspected organism is in fact the organism which was stolen, is quite difficult.

Second, even though serious problems result from overuse or misuse of organisms, the origin thereof cannot be clearly found. damage. That is, when, instead of organisms a user wants, organisms similar thereto are supplied to the user due to supplier error, it is impossible to clearly distingush whether or not the supplied organisms are the same as those that were ordered. Accordingly, the user may suffer from serious time and economic loss.

Due to these problems, there is a need for a method for marking inherent information present inside organisms. Due to their inherent characteristics, marking information into organisms is not easy.

Meanwhile, Korean Patent Laid-open N. 1999-0074315 (published on Oct. 5, 1999) discloses a method for marking microorganisms using a DNA sequence, the method comprising: making a DNA sequence which corresponds to an English string of a desired name tag; ligating the DNA sequence to make a series of character strings; attatching a primer for polymerase chain reaction (PCR) to both sides of the character string and a restriction site, enabling ligatation with a suitable vector, to the outside thereof; treating the restriction site with a restriction enzyme to make a sticky end; inserting the DNA sequence into a vector treated with the same restriction enzyme; and transforming a target microorganism with the vector, wherein by attatching tags to the organism wastes of biological contaminants or marking an English name of an organization which developed the organisms, the English name can be found, the origin of the wastes can thus be established, and furthermore, by tagging plasmids or vectors into which novel genes are cloned, the owner of the microorganism can be clearly expressed and the occurrence of dispute associated with the illegal use of microorganisms can thus be prevented.

The key of the pulished patent is that the DNA sequence corresponding to the English string of the desired name tag is inserted into a vector, and the resulting vector is then inserted into microorganisms to transform the microorganisms.

However, the pulished patent has the following serious problems:

First, when microorganisms in which vectors (or plasmids) are contained in the cytoplasm thereof are transformed, the vectors may be excluded therefrom, as the cell culutre proceeds. This cell cultivation causes the problem wherein markers cannot be present in microorganisms any more.

Second, in the case where microorganisms in which vectors present therein are intentionally excluded are used, the owner of the microorganisms cannot be identified.

Third, like the method of the published patent, when vectors (plasmids) are used to transform microorganisms, vector systems that can operate in the corresponding microorganisms must be provided. However, only a few species of microorganisms with host-vector systems are available and thus this method cannot be generally used.

Fourth, actually commertially available strains are generally obtained by mutation and vector systems for these transformed strains are not well developled. In this regard, the published patent is disadvantageously inapplicable to conventional useful strains.

### DISCLOSURE OF INVENTION

### Technical Problem

It is an object of the present invention to provide a new method that is broadly applicable to all biological organism and can prevent intentional exclusion of bio-information markers.

### Technical Solution

In accordance with one object of the present invention for achieving the above object, there is provided a method for marking bio-information into an organism by inserting inherent bio-information encoded to a DNA sequence or RNA sequence into genome of an organism through a gene delivery system.

### ADVANTAGEOUS EFFECTS

According to the present invention, inherent bio information is inserted into genome of organisms, thus avoiding loss by cultivation or artificial manipulation and being widely used for even organisms for which a host-vector system is not available.

Based on these features, the method has the following advantages. First, inherent bio-informtion can be clearly obtained from the organisms themselves, rather than an additional means such as catalogs. Second, when organisms developed through extensive efforts are stolen, they can be tracked and identified. Third, when serious problems occur by overuse or misuse of organisms, the origin thereof can be clearly identified.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a schematic diagram illustrating a summary of the present invention;

FIG. 2 is an example of a recognition table of the present invention; and

FIG. 3 is a process for inserting inherent bio information encoded to a DNA sequence into genome of organisms through a transposon system.

### BEST MODE FOR CARRYING OUT THE INVENTION

Hereinafter, the present invention will be described in more detail.

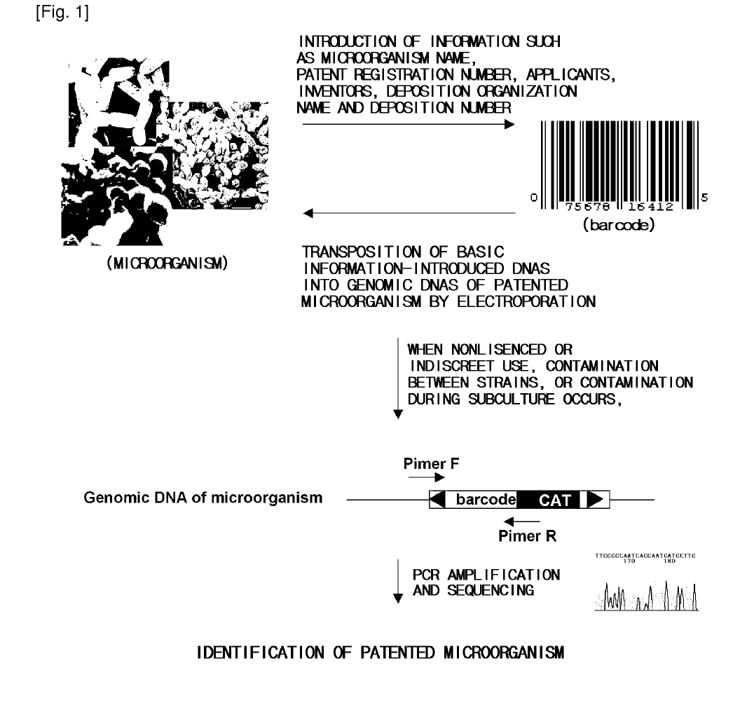

The present invention provides a method for marking bio-information, comprising inserting inherent bio-information encoded to a DNA sequence or RNA sequence into genome of organisms through a gene delivery system (See FIG. 1).

The method according to the present invention is characterized in that inherent bio-information encoded to a DNA sequence or RNA sequence is inserted into genome of organisms. When inherent information is inserted into genome of organisms, bio-information encoded to the nucleotide sequence is introduced into the genome. Accordingly, such a method has an advantage in that the introduced genes are not lost and are stably maintained, as compared to conventional gene transformation technology using plasmids. That is, plasmid shuttle vector systems have a disadvantage of low segregational stability, thus causing a serious vector loss problem upon repetition of generation. On the other hand, the method of the present invention is advantageously free of the afore mentioned problems, because a specific nucleotide sequence is inserted into the genome of host cells.

In addition, in the case of plasmid vectors, the corresponding vectors can be intentionally excluded to the outside of strains. According to the present invention, by randomly and secretly inserting bio information encoded to a specific nucleotide sequence, into genome of organisms, the malicious behaviors can be prevented.

Meanwhile, according to the present invention, inherent bio information encoded to sequences of DNA or RNA is inserted into genome of organisms. The DNA sequence can be inserted into genome through a conventional gene delivery system without any additional consideration. However, when the RNA sequence is used, a reverse transcription enzyme to convert the RNA sequence into DNA sequence must be taken into consideration. At this time, any method well-known to those skilled in the art may be used.

The inherent bio information as used herein may be a variety of useful information associated with organisms. Examples of information can include a depositor or an owner, inventors, a deposition date, main transformation elements and directions upon use.

The gene delivery system as used herein is characterized in that bio-information encoded into a specific nucleotide sequence is inserted into genome. Accordingly, any method well-known in the field of genetic engineering may be used so long as it can insert foreign genes into genome present in hosts(for example, bacteria) In particular, a transposon system may be used. The transposon system is a method to randomly introduce specific genes into the genome of hosts, which is applicable to all microorganisms, animal cells and plant cells (Insect Molecular Biology (2007), 16(1), 37-47, Plant Physiology Preview. Published on Nov. 9, 2007, as DOI:10.1104/pp. 107.111427, the American Society of Plant Biologists; research on production of lactoferrin from transformed silkworms and functionality thereof, the Ministry of Agriculture and Forestry, 2005)

According to the present invention, when the transposon system is used, even experimenters cannot find the place of the genome into which the nucleotide sequence is inserted. When the position of specific information encoded to the nucleotide sequence is not exposed, no one can find the insertion position, thus advantageously causing the impossibility of arbitary deletion.

Those who insert the nucleotide sequence into genome know the specific neleotide sequence of templates to which the specific primers are attatched upon PCR. Accordingly, obtaining bio information encoded to the nucleotide sequence through PCR has no problem. The inherent bio information is classified into information which can open and information which cannot open, encoded to a plurality of nucleotide sequences of DNA or RNA that are independent of one another, and is then inserted into genome of organisms, thereby controlling the target and level to be opened. That is, PCR primers for information that is open to the public may be available, whereas PCR primers for confidential information may be kept secret, thereby controlling the targets and levels to be opened.

Meanwhile, due to the possibility of a gene knock-out by random insertion of bio-marking sequence into genome, confirmation for non-alteration of physiological characteristics of strain should be followed. The scope of confirmation generally depends on the depositary's interest. Generally and primarily, cell growth rates are compared between transformants and host cells. Further biochemical comparison/identification methods are useful for the comparison and API kit system is generally used. The depositary can choose a transformant which has no alteration in its genomic and physiological characteristics by using appropriate methods. For a convinced result, genomic library can be made and CM gene fragment can be cloned in E. coli for further sequencing and confirmation of non knock out of any ORF in the genomic DNA.

Meanwhile, the antibiotic chloramphenicol-resistant marker as used herein may be designed such that marker genes can be removed, if necessary, when in the process of forming a transposon system, a recognition site (nucleotide sequence) of a Cre or Flp recombinase is inserted into both ends of the marker genes.

When marker genes of yeasts are URA3, repetitive nucleotide sequences are inserted into both ends thereof to perform counter selection using 5′-FOA. As a result, advantageously, the URA3 genes can be repetedly used as markers. Advantageously, a new bar code containing other information can be introduced into genome of marker-removed host cells using a transposon system containing another bio-marking.

The method for marking bio-information using a transposon system according to the present invention randomly inserts bio-information into genome of most host cells. For this reason, the method has potent advantages in that it can target almost all host cells including unknown strains whose biochemical or genetic information is unknown and, in particular, can be used for strains for which a gene recombination system has not been established.

Suitable organisms include bacteria, molds, insects, animal cells, animals, plant cells and plants. All of these organisms have genome and any known gene delivery system (e.g., transposon system) enabling random insertion of specific nucleotide sequences into the genome may be used. At this time, bio-information encoded to a specific nucleotide sequence can be introduced by a method for transplanting animal or plant cells into which bio information encoded to a specific nucleotide sequence is introduced.

Furthermore, the present invention provides an organism in which inherent bio-information is inserted into genome, obtained by the method according to claim 1. For example, the organism may be selected from bacteria, molds, insects, animal cells, animals, plant cells and plants.

The present invention provides a method for reading bio information from a bio-information-marked organism, comprising: amplifying, from an organism in which inherent bio-information encoded to a DNA sequence is inserted into genome thereof, obtained by the method according to claim 1, the encoded DNA sequence through PCR to obtain PCR products; analyzing a nucleotide sequence of the amplified PCR product; and decoding the nucleotide sequence by comparison with the decoding table.

The method of reading bio-information is that an encoded DNA sequence is read through PCR from an organism, in which inherent bio-information encoded to the DNA sequence has been inserted into genome, obtained by the method according to claim 1.

The primers used for PCR include forward and reverse primers. When inherent bio-information is encoded to a DNA sequence, the primers are complementary to the nucleotide sequences present at the 5′ and 3′ ends of the DNA region. The primer sequence can be recognized by designing inherent bio information into a nucleotide sequence and is already known to those who design inherent bio information to a DNA sequence or obtain it from the DNA sequence.

The forward and reverse primers to specific templates can be readily produced by a method well-known to those skilled in the art, and a detailed explanation thereof will be thus omitted (Dieffenbach C W, Dveksler G S. 1995. PCR primer: a laboratory manual, New York, N.Y.: Cold Spring Harbor Laboratory Press; New England Biolabs Inc., 2007-08 Catalog & Technocal Reference)

In the method for reading bio-information, primers for PCR may be a plurality of sets of forward and reverse primers that operate independently from each other. In this case, some primers not open to the public cannot be subjected to PCR by those except for designated persons and specific information thereof cannot be read. On the other hand, the other primers open to the public can be subjected to PCR by any one and specific information thereof can be thus read by everyone. As a result, targets and levels to open can be controlled according to the type of information.

The method comprises, after obtaining PCR product, analyzing a nucleotide sequence of the amplified PCR product. This step is to analyze, so-called “to sequence” the nucleotide sequence of the amplified PCR product. The sequencing is carried out using a method well-known to those skilled to the art. Sequencing technologies are generally known, for example, there are many commercial sequencing service providers.

The method for reading bio-information comprises, after analyzing the nucleotide sequence of amplified PCR products, decoding the nucleotide sequence by comparison with a recognition table. In this step, the analyzed nucleotide sequence is decoded by comparison with an additionally provided recognition table. The decoding may be carried out by a software.

### MODE FOR THE INVENTION

Hereinafter, the present invention will be explained in more detail with reference to the following examples. The scope of the invention is not necessarily limited to these examples and incorporates modifications with equivalent technical ideas.

### EXAMPLES

### Example 1

### Production of Bio-Information Introduced Leuconostoc citreum Strains

\textcircled{1} Determination of Specific DNA Sequence

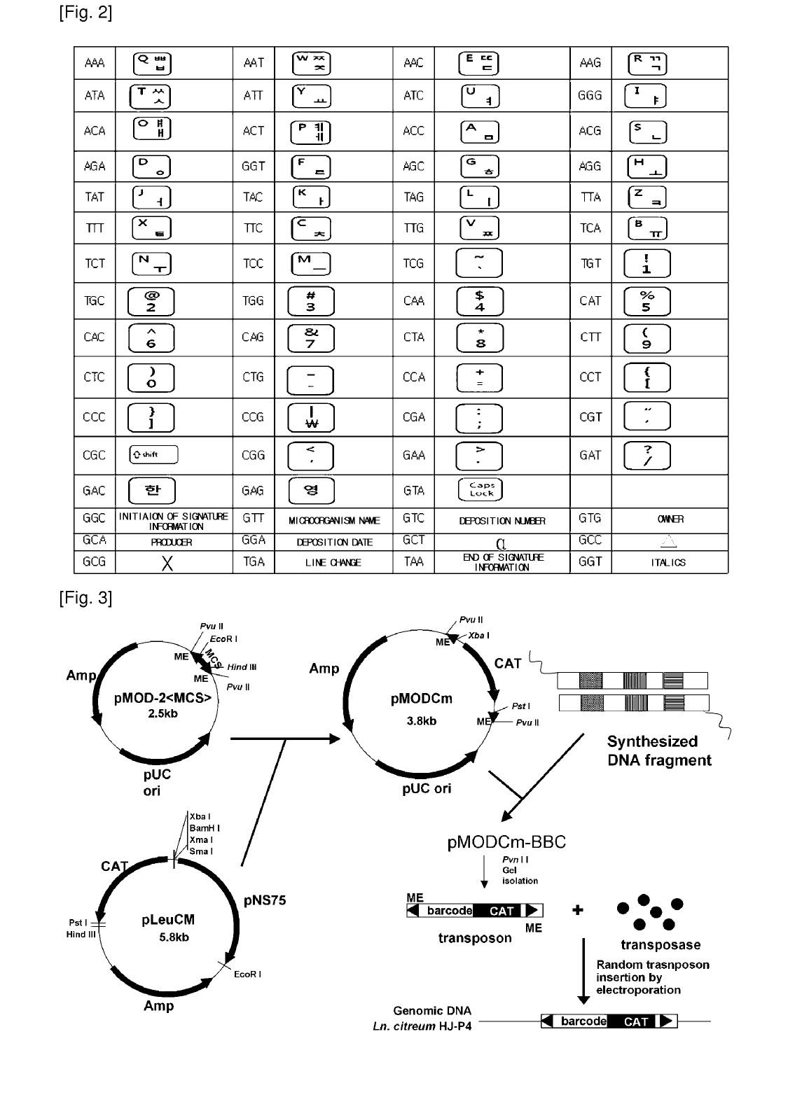

First, inherent bio-information was encoded to a DNA sequence according to a code table (recognition table) shown in FIG. 2 and a total of 117 by (39×3) sequences were determined and ligated.

Strain name: Ln. citreum

GGCGTTTAG TCT GAA TTC GGG ATA AAG ATC TCC

(Initiation of signing) (Strain name) (Ln. citreum)

Patent Registration No.: 560160

CAT CAC CTC TGT CAC CTC

(560160)

Owner: CBNU

GTGTTC TCA TCT ATC

(Owner) (CBNU)

Inventor: HAN NS

GCAAGG ACC TCT TCT ACG

(Inventor) (HAN NS)

Depositary and Deposition No.: KACC 91035

TAC ACC TTC TTCGTCCTT TGT CTC TGG CATTAA

(KACC) (Deposition Nb.) (91035) (End)

\textcircled{2} Establishment of Transposon System

The 117 by nucleotide sequences determined as above (hereinafter, refered to as a “strain information sequence”) were inserted together with selective marker genes into vectors for eatablishing transposon system.

The pMOD-2<MCS> vector as used herein was obtained from Epicentre (Madison, Wis., USA). This vector contains pUC-ori as a basic component and is used for cloning of PCR product of a target gene to be transpositioned in a multiple cloning site (MCS), or for obtaining fragments containing both mosaic ends (ME) with specific restriction enzymes (Pvu II and PshAI, these two restriction enzymes can be used only when fragments cloned therewith are not cut)

In the present experiments, Leuconostoc citreum (Deposition N. KACC 91035), superior starter strain, which had been isolated in Kimchi and is currently used as a starter to produce Kimchi, was used as host cell, and chloramphenicol-resistant gene (CAT) which endow a resistance against chloramphenicol in lactic acid bacteria was used as selective marker.

First, pLeuCM (shuttle vector can be inter-cloned in Leuconostoc citreum and E. coli; Korean patent registration N. 0721140) was treated with PstI and XbaI restriction enzymes, and purified through an agarose gel electrophoresis to obtain chloramphenicol-resistant gene (CAT) fragment.

Then, pMOD-2<MCS> vector was also treated with the said restriction enzymes, PstI and XbaI and were ligated with the obtained chloramphenicol-resistant gene (CAT) and introduced into an Escherichia coli (E. coli) Top10 and the vector called pMODCm was finally obtained through isolation.

Subsequently, the strain information sequence ligated as above was inserted into a cloning site interposed between both ME sites of pMODCm vector.

The general contents as afore-mentioned can be seen in FIG. 3.

\textcircled{3} Transposition

The pMODCm vector obtained in section \textcircled{1} contain 19 by mosaic end (ME) sites where transposition was induced by recognition of a transposase available from Epicentre and were treated with PCR or PvuII restriction enzymes to obtain ME-site containing DNA fragments.

The present inventors obtained DNA fragments by treating the pMODCm vector with PvuII restriction enzymes. The DNA fragments (containing chloramphenicol-resistant gene (CAT) and ME sites) 2 μl [100 mg/ml in a TE buffer (10 mM Tris-HCl, pH 7.5), 1 mM EDTA] were mixed with EZ::TN transposase (Epicentre, Madison, Wis., USA) 4 μl and glycerol 2 μl and the reaction was proceeded at room temperature for 30 minutes. 1 μl of the resulting product was used for each transformation.

Competent cells (40 μl) of the host, Leuconostoc citreum strains were made, transferred with the resulting product thus obtained to a cuvette and then placed in an ice bath for 5 minutes. Immediatedly after an electric pulse was applied at 25 μF, 8 kV/cm, 400 ohms, 1 ml MRS liquid medium was added thereto and incubated at 30° C. for approximately one hour. Then, the resulting culture medium was spreaded on a 10 μg/ml chloramphenicol-containing MRS plate and incubated at 30° C. for 48 hours. Then, transformed cells were selected.

\textcircled{4} Confirmation of Transposition

A number of chloramphenicol (CM)-resistant Leuconostoc citreum colonies were obtained from the section \textcircled{3} and then cultivated in a chloramphenicol-containing MRS medium for one hour.

In order to confirm whether chloramphenicol-resistant gene (CAT) was inserted into the genome of Leuconostoc citreum, genomic DNAs were isolated from the strains and chloramphenicol-resistant gene was identified by PCR using primers.

The isolation of genomic DNAs was carried out by using AccuPrep Genomic DNAs Extraction Kit' (available from Bioneer, Inc.) as follows. First, a wild-type Leuconostoc citreum as a contol group and strains selected in the present Example were incubated in an MRS medium and a chloramphenicol antibiotic-containing MRS medium, respectively, and the resulting media were centrifuged at 13,000 rpm for 2 minutes to collect cells. The cells thus collected were washed with a TES solution (30 mM Tris-HCl, 50 mM NaCl, 5 mM EDTA, pH 8.0), suspended with addition of 100 μl of a 6.7% sucrose (50 mM Tris, 1 mM EDTA, pH 8.0) solution and incubated at 37° C. for 30 minutes. 10 mg/ml of a lysozyme in 100 μl of 25 mM Tris buffer (pH 8.0) was added to the cells and cultured at 37° C. for 30 minutes. The following process was carried out in accordance with the protocol to obtain a final pure product (50 μl)

The purified genomic DNAs were used as PCR templates, and the reaction mixture (50 μl) was composed of Taq DNA polymerase 0.5 μl, a 10×buffer, 250 μM dNTPs, and primers CM-For: 5′-CATATCAAATGAACTTTAAT-3′, CM-Re: 5′-ATCTCATATTATAAAAGCCA-3′. PCR conditions were as follows: denaturation: 94° C., 5 minutes; standard PCR 30 cycles: 94° C., 30 seconds/55° C., 30 seconds/72° C., 1 minute; final reaction: 72° C., 5 minutes.

The experimental results ascertained that chloramphenicol-resistant genes were suitably inserted into the genome of selected strains.

\textcircled{5} Amplification and Sequencing of Strain Information Sequence by PCR

After genomic DNAs of the selected strains were collected, PCR was performed. The primers as used herein were forward primers designed to be complementarily bound to a 5′-GGC GTT TAG TCT GAA TTC-3′ position of templates, and reverse primers designed to be complementarily bound to a 5′-CTT TGT CTC TGG CAT TAA-3′ position of templates.

As a result of PCR, PCR products having about 120 by nucleotide sequences can be obtained and subjected to sequencing. The PCR results ascertained that the PCR products exactly corresponded to the strain information sequence.

The sequence of the PCR product was again compared with the decoding table shown in FIG. 2. The comparison results ascertained the sequence of the PCR product exactly corresponds to the items which were intended to be initially marked.

1. A method for marking bio-information, by inserting into genomic DNAs of an organism through a transposon system (a) a first DNA or RNA base sequence representing bio-information inherent to the organism, and (b) a second DNA or RNA base sequence capable of complimentarily binding to at least one of a forward primer or a reverse primer so as to enable the first base sequence to be cloned by PCR.

2. (canceled)

3. The method according to claim 1, wherein the inherent bio-information is encoded to a plurality of DNA base sequences or RNA base sequences independent from each other.

4. The method according to claim 1, wherein the organism is selected from bacteria, molds, insects, animal cells, animals, plant cells and plants.

5. An organism in which bio-information is inserted in the form of a DNA base sequence into genomic DNAs, obtained by the method according to claim 1.

6. The method according to claim 5, wherein the organism is selected from bacteria, molds, insects, animal cells, animals, plant cells and plants.

7. A method for reading bio-information from an organism having genomic DNAs that comprise (a) a first DNA base sequence representing bio-information inherent to the organism, and (b) a second DNA base sequence capable of complimentarily binding to at least one of a forward primer or a reverse primer so as to enable the first base sequence to be cloned by PCR, comprising:
amplifying the first base sequence by PCR to obtain a PCR product;
analyzing the base sequence of the amplified PCR product; and
comparing the analyzed base sequence with a recognition table to decode the base sequence.

8. The method according to claim 7, wherein the PCR uses, as primers, a plurality of sets of forward and reverse primers independent from each other.

9. The method according to claim 7, wherein the decoding is carried out by a computer program.
